(19) United States
(12) Patent Application Publication
(10) Pub. No.: US 2023/0363637 A1
(43) Pub. Date: Nov. 16, 2023

(54) REMOTE SUBJECTIVE REFRACTION TECHNIQUES

(71) Applicant: DigitalOptometrics LLC, Lake Success, NY (US)

(72) Inventor: William K. Van Cleave, Abilene, TX (US)

(21) Appl. No.: 18/198,111

(22) Filed: May 16, 2023

Related U.S. Application Data
(60) Provisional application No. 63/342,421, filed on May 16, 2022.

(51) Int. Cl. A61B 3/028; G16H 80/00; G16H 10/60; G16H 40/67

(52) U.S. Cl. CPC A61B 3/028; G16H 80/00; G16H 10/60; G16H 40/67

(57) ABSTRACT

Systems, apparatuses, and techniques are disclosed for enabling a remote vision examination for a user accessing a system having a user device, a display, and a sensor. In some implementations, a communication session is established over a network between the user device and at least a first provider device included in one or more provider devices. Calibration data is obtained from the user device during the remote session. An egocentric distance is determined between the user and the user device based on the calibration data. A determination that a set of criteria are satisfied is made based on the egocentric distance and the calibration data. A remote vision examination is administered in response to the determination that the set of criteria are satisfied.

### CROSS-REFERENCE TO RELATED APPLICATIONS

This application claims the benefit of U.S. Provisional Application No. 63/342,421, filed May 16, 2022, the disclosure of which is incorporated herein by reference in its entirety.

### TECHNICAL FIELD

This disclosure relates generally to remote comprehensive eye examinations, and more specifically, to subjective refraction examinations.

### BACKGROUND

Subjective refraction is a technique to determine the combination of lenses that will provide the best corrected visual acuity (BCVA). A subjective refraction examination is a clinical examination used by orthoptists, optometrists and ophthalmologists to determine a user's, i.e., patient's need for refractive correction, in the form of glasses or contact lenses.

Various equipment is used to conduct a subjective refraction test. This typically requires trial frames, trial lens box and confirmation set (pinhole and occluder), and a Snellen chart. The equipment can also include a Jackson Cross Cylinder, which is a combination of two cylinders whose powers are numerically equal and of opposite sign (+/−) and whose axis are perpendicular to one another. This can be used to search for astigmatism. A Duochrome test can also be used to check the spherical component of the refraction.

Subjective refraction generally consists of three distinct phases. The first phase is designed to correct the spherical element of the refractive error in such a way as to facilitate the accurate determination of any astigmatic element present. The second phase is the determination of the astigmatic error and the third phase involves the balancing and/or modification of the refractive correction to ensure optimal visual performance and patient comfort. The user's history and symptoms can be used to help predict a refractive error.

### SUMMARY

This disclosure focuses on systems, apparatuses, and techniques for facilitating remote vision examinations over distributed computing systems hosted on wide area networks. The computing systems disclosed herein leverage visual sensors and displays, which, in some instances, may be consumer-grade devices available to many users to reduce system complexity and overall user burden. The systems described herein further enable techniques for calibrating, processing, and optimizing sensor data collected by user devices (e.g., cameras, displays) to produce usable data for remote vision examinations with reasonable accuracy without necessitating the use of specialized ophthalmic equipment and/or the user to be examined in a specialized location (e.g., diagnostic center).

The systems disclosed herein further leverage computer vision and machine learning techniques to aid a vision service provider (e.g., orthoptist, optometrist, ophthalmologist, refractionist) to efficiently evaluate, diagnose, and treat various conditions and diseases related to the eyes and vision without being co-located with a patient and/or without requiring the patient to have access to specialized ophthalmic equipment. In this way, the systems and techniques enable patients in locations without adequate access to vision service providers and/or to still receive treatment, thereby increasing the overall access to eye care services.

For example, in some implementations, the systems enable a vision service provider to conduct a subjective refraction test for a patient while the patient is located at home and using devices and materials already available to the user. In such implementations, a vision service provider interacts with the user through an audio or video conference. The vision service provider instructs the user to adjust his/her device to emulate the lens adjustments that would be performed during a traditional subjective refraction test.

As discussed herein, the systems further enable calibration procedures prior to administration of remote vision examinations to ensure accuracy of collected examination data and/or a prescription generated based on the collected examination data. A cloud-based service (e.g., implemented on a server) can receive images of the user in real-time (or substantially in real-time). In some implementations, the server may be configured to apply one or more neural networks to identify facial landmarks (e.g., corneal perimeter) to compute a distance between a user's eyes and a display screen used to provide visual information to the user during a remote vision examination. In such implementations, data collected from calibration procedures thereby combines with outputs of neural networks to render a customized eye chart on the user's display in near real-time, which is then used to administer a remote vision examination.

As described herein, an “egocentric distance” refers to a distance between an observer and an external point in space. In vision examinations, the egocentric distance can be used as a measure of visual perception and spatial awareness. By accurately perceiving distances, users are able to navigate their environment safely and effectively. Tests of egocentric distance perception may involve asking individuals to judge the distance of objects or to make reaching movements towards objects at varying distances.

As described herein, a “vision examination” refers to an evaluation of a user's visual abilities and health. Vision examinations are typically performed by vision service providers, such as an orthoptist, optometrist, ophthalmologist, or a refractionist. A variety of tests and/or techniques may be used by a vision service provider during a vision examination to assess a user's (or patient's) vision. As examples, during a vision examination, a vision service provider may perform tests to assess visual acuity, depth perception, color vision, eye alignment and movement, and peripheral vision. The vision service provider may also check the health of the eyes by examining the retina, optic nerve, and other structures using instruments such as a slit lamp and ophthalmoscope. The results of a vision examination can help identify potential vision problems, such as nearsightedness, farsightedness, astigmatism, or other refractive errors. A vision examination can also identify other eye conditions, such as cataracts, glaucoma, or age-related macular degeneration, that may require further treatment or management. Based on the results of the vision examination, the vision service provider may recommend corrective lenses, vision therapy, or other treatments to improve or manage the user's visual health and well-being The systems and techniques discussed throughout this disclosure facilitates administration of “remote” visual examinations, where a vision service provider is not co-located with a user as the examination is being administered. Further, using the techniques described herein, a user may receive a remote vision examination without needing access to specialized ophthalmic equipment (e.g., phoropter) such that he/she does not travel to a diagnostic facility for the remote vision examination to be performed.

As described herein, “real-time” refers to information or data that is collected and/or processed instantaneously with minimal delay after the occurrence of a specified event, condition, or trigger. For instance, “data collected in real-time” or “real-time data” refer to data, e.g., sensor data used for calibration of a camera and/or display, etc., that is processed with minimal delay after a computing device collects or senses the data, e.g., using accelerometers, gyroscopes, magnetometers, etc. The minimal delay in collecting and processing the collected data is based on a sampling rate or monitoring frequency of the computing device, and a time delay associated with processing the collected data and transmitting the processed data over a network (e.g., using one or more neural networks to identify facial landmarks).

In one general aspect, this disclosure involves a method of enabling a remote vision examination for a user over a wide area network. The method includes obtaining, from one or more user devices, calibration data relating to a user positioning for the remote vision examination. An egocentric distance is determined between the user and the one or more user devices based on the calibration data. The method further includes determining that a set of constraints associated with the remote vision examination have been satisfied at least on based on the egocentric distance and the calibration data. In response to determining that the set of constraints have been satisfied, the method includes transmitting, to a provider device, an instruction that permits a vision service provider to administer the remote vision examination. A communication session is established over the wide area network between the one or more user devices and at the provider device. Examination data generated during the remote vision examination is collected. The examination data is transmitted to the provider device in a manner that permits the vision service provider to at least evaluate the examination data.

One or more implementations may include the following optional features. For example, in some implementations, the one or more user devices include a computing device that displays information relating to remote vision examination and a camera for collecting video data of the user during the remote vision examination. In such implementations, the calibration data includes a focal length of a camera, and one or more distortion coefficients associated with radial distortion of a lens of the camera.

In some implementations, the calibration data includes a size associated with a reference object.

In some implementations, the examination data includes information associated with a subjective refraction test.

In some implementations, the examination data includes information related to a subjective refraction. In such implementations, transmitting the examination data to the provider device permits the vision service provider to determine a best-corrected visual acuity for the user based on the examination data.

In some implementations, the remote vision examination includes a visual acuity test.

In some implementations, the one or more user devices include a computing device that displays information relating to remote vision examination and a camera for collecting video data of the user during the remote vision examination. In such implementations, the set of constraints includes a physical coupling the computing device and the camera, a user device configuration in which a sensor of the user device is on a same plane as a display of the computing device, a network connection speed having an upload or download speed of at least about 50 Mbps, and a minimum ambient light level of about 500 lux.

In some implementations, the set of constraints include a trained reference object detector capable of facial landmark recognition is installed on a server coupled to the wide area network.

In some implementations, the method further includes determining that information specified by the calibration data does not satisfy one or more constraints included in the set of constraints. In such implementations, in response to determining that the information does not satisfy the one or more constraints, the method includes determining a set of actions to be performed by the user. The method further includes obtaining, from the one or more user devices, second calibration data, where the second calibration data indicates that the user has performed the set of actions. Further, a determination is made that the information specified by the second calibration data satisfies the set of constraints.

In some implementations, in response to determining the egocentric distance, the method includes providing, to the one or more user devices, an instruction to display one or more optotypes having a width and height that corresponds to the egocentric distance.

In some implementations, the method includes determining a pixel density of a display included in the one or more user devices. In such implementations, the method includes determining a minimum angle of resolution for the one or more optotypes for display on the display device, and transmitting, to the provider device, information related to a calibration of the display.

Implementations of the described techniques can include hardware, a method or process implemented at least partially in hardware, or a computer-readable storage medium encoded with executable instructions that, when executed by a processor, perform operations. The details of one or more implementations are set forth in the accompanying drawings and the description below. Other features will be apparent from the description and drawings.

### DESCRIPTION OF DRAWINGS

FIGS. 1A-1B illustrates an example of a system for facilitating remote vision examinations. FIG. 1A illustrates examples of elements of the system. FIG. 1B illustrates an example of technique for facilitating a remote vision examination.

FIGS. 2A-2C illustrate examples of processes for administering remote vision examinations. FIG. 2A illustrates an example of a process for administering a remote vision examination using automated egocentric distance calibration. FIG. 2B illustrates an example of a process for administering a remote vision examination using manual egocentric distance calibration. FIG. 2C illustrates an example of a process for calibrating user devices for a remote vision examination.

FIGS. 3A-3F illustrate examples of user interfaces during one exemplary remote subjective refraction examination.

FIGS. 4A-4H illustrate examples of user interfaces during a second exemplary remote subjective refraction examination.

In the drawings, like reference numbers represent corresponding parts throughout.

### DETAILED DESCRIPTION

In general, systems, apparatuses, and techniques are described for a computer system capable of administering a remote vision examination (e.g., a subjective refraction test administered in a user location that is remote from a provider location). The systems disclosed herein enable the provider (e.g., orthoptists, optometrists, ophthalmologists) to perform the remote subjective eye refraction without requiring the user and the vision healthcare provider to be in the same location and/or without requiring the user to have any specialized equipment at the test location. In some implementations, the computer systems enable a user to have a remote subjective eye refraction test at a user's home using devices and materials already available to the user. In such implementations, a vision healthcare provider interacts with the user through an audio or video conference. The vision healthcare provider instructs the user to adjust his/her device to emulate the lens adjustments that would be performed during a traditional subjective refraction test.

The present disclosure makes use of a unique combination of calibration methods, facial landmark detectors, and display features to bring visual acuity tests directly into a user's home. When a trained neural network is combined with sensor calibration techniques, the system uses triangulation to derive an absolute location of reference objects with respect to the camera. Given an unknown test environment (the user's home), an unknown display, and an unknown camera, we need to apply a set of constraints programmatically to assure the accuracy of the near real-time subject-to-camera distances. A validation object can be used to further refine calibration results obtained by utilizing the reference object.

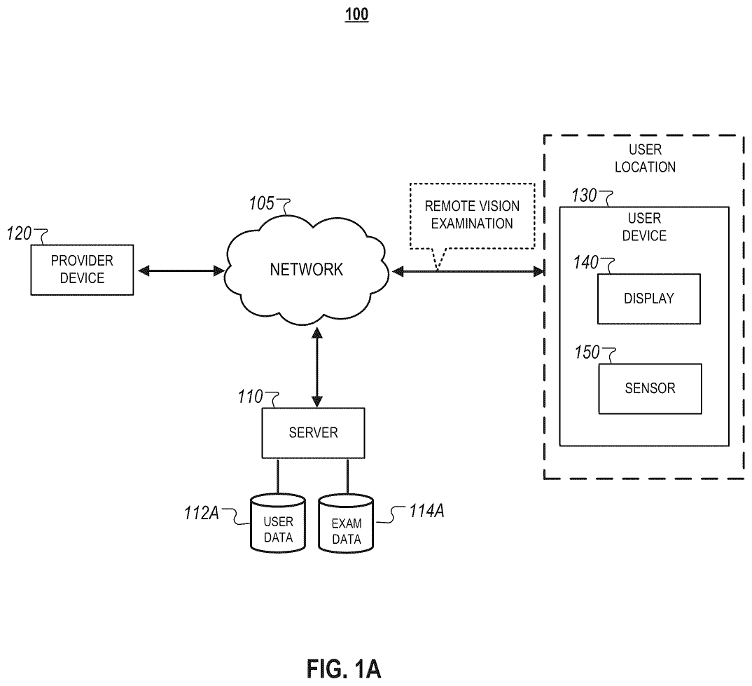

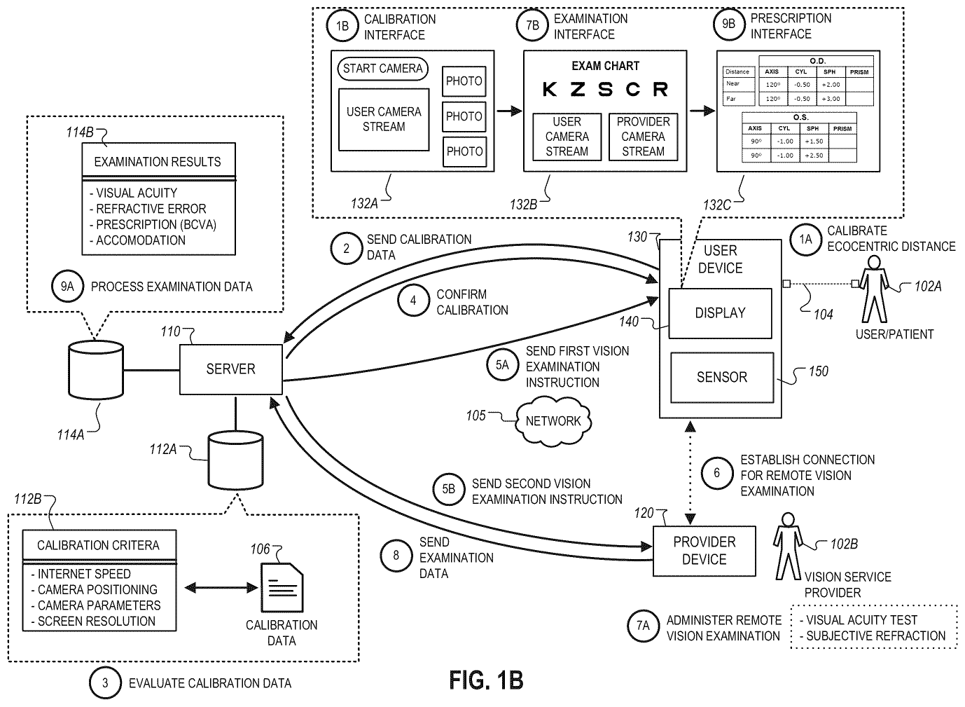

FIG. 1A illustrates an example of a system 100 that can be used to facilitate remote vision examinations. The system 100 includes a server 110, a provider device 120, a user device 130, a display 140, and a sensor 150 which communicate over network 105. The server 110 stores data 112A and data 114A. In some implementations, two or more of the user device 130, the display 140, and the sensor 150 can be coupled in a distributed arrangement where user device 130, display 140, and sensor 150 is coupled through the network 105. In another implementation, the display 140 and the sensor 150 can be embedded into, i.e., physically and electrically coupled to, the user device 130.

As described throughout, the system 100 enables remote vision examinations (e.g., subjective refraction examination) provided by system 100, which allows a vision service provider 102B (e.g., optician, optometrist, ophthalmologist) to be in a location that is remote from a location of the user 102A (e.g., patient) undergoing examination by the provider 102B. This is enabled by techniques disclosed herein, such as calibrating display 140 such that adjustments by the user 102A during the examination emulate the use of trial lenses of a dedicated subjective refraction unit in a physical location, such as a doctor's office. Advantageously, the system 100 thereby enables the provider 102B to perform subjective refraction from any suitable location that is capable of connecting to the server 110 via the network 105. Moreover, the system 100 enables the user 102A similar benefits by not requiring the user 102A to travel to a specialized location to be examined. For example, other remote eye examination techniques require the user 102A to be co-located with a specialized ophthalmic unit, such as a phoropter, in order to implement the co-located examination technique. Existing methods for performing visual acuity exams at home require that the patient measures their own distance to the exam chart. This operation introduces both user error and increases the overall difficulty of the exam. Another advantage of the system 100 disclosed herein, is that the server 110, implementing the methods disclosed herein, allow the user 102A can configure his/her user device 130 so that he/she can receive a vision examination in any convenient location, such as his/her home. Another advantage of the disclosed system is that it replaces the need for a subjective refraction unit, which can cost thousands to tens of thousands of dollars, and has large space requirements and requires special maintenance.

Referring now to components of system 100, the network 105 can be configured to enable electronic communications between electronic devices, such as the server 110, user device 130, display 140, and the sensor 150. For example, the network 105 can be configured to enable exchange of electronic communications between server 110, provider device 120, user device 130, and provider device 120. Network 105 can include Local Area Networks (LANs), Wi-Fi, or analog or digital wired and wireless networks (e.g., ZigBee, Z-wave). Network 105 can include multiple networks or subnetworks, each of which can include, for example, a wired or wireless data pathway. Network 105 can also include a circuit-switched network, a packet-switched data network, or any other network able to carry electronic communications (e.g., data or voice communications). For example, network 105 can include networks based on the Internet protocol (IP), or other comparable technologies. In some examples, the network 105 can include wide area networks (WAN) of computers that receive services provided by provider 102B.

Server 110 can be an electronic device configured to provide services for remote vision examinations conducted over network 105. Server 110 can be configured to monitor events related to a vision examination, such as a user requesting a vision examination, the start/end of a remote communication session between provider device 120 and user device 130, generation and/or collection of examination data by a provider, submission of a best-corrected visual acuity by the provider, among others. Server 110 can also implement processes relating to intermediating communications between provider device 120 and user device 130, such as selecting a provider from a set of available providers to remotely perform a remote vision examination, processing communication data received from provider device 120 and user device 130 during an ongoing remote vision examination, processing input data provided by the user 102A during the remote vision examination, among others. Server 110 stores data 112A, which includes information associated with user device 130, and data 114A, which includes information relating to a remote vision examination.

The provider device 120 can be an electronic device of a provider that conducts a remote vision examination. Provider device 120 permits a provider to access a remote communication session with user device 130 over network 105 and through which the provider can perform a remote vision examination. For example, either the provider device 120 or the user device 130 can be one or more of a desktop computer, server computer, portable computer, smart phone, tablet computer, game console, set top box, media player, smart TV, and the like.

In some implementations, the remote communication session is a synchronous communication session, such as an audio conference or a video conference, where the provider and the user 102A communicate in real-time (or substantially in real-time). In such implementations, the provider may communicate instructions to the user 102A to adjust the user device 130 and/or the display device to simulate a different trial lens configuration. The provider may then obtain user feedback relating to vision, which can be used to determine a best corrected visual acuity. In other implementations, the remote communication session is an asynchronous communication in which a user performs a predetermined set of actions (e.g., representing different lens configurations and corresponding vision feedback) relating to the user device 130 and the display 140. In such implementations, the provider may use previously generated data to determine a best corrected visual acuity, and if necessary, conduct any follow-up with the user 102A (e.g., requesting a synchronous communication session since the user data may not be sufficient to determine a best corrected visual acuity).

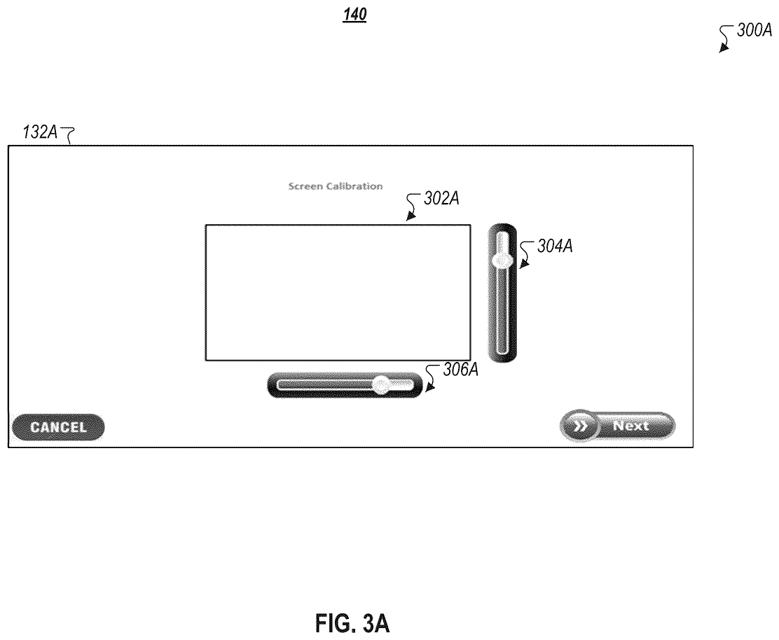

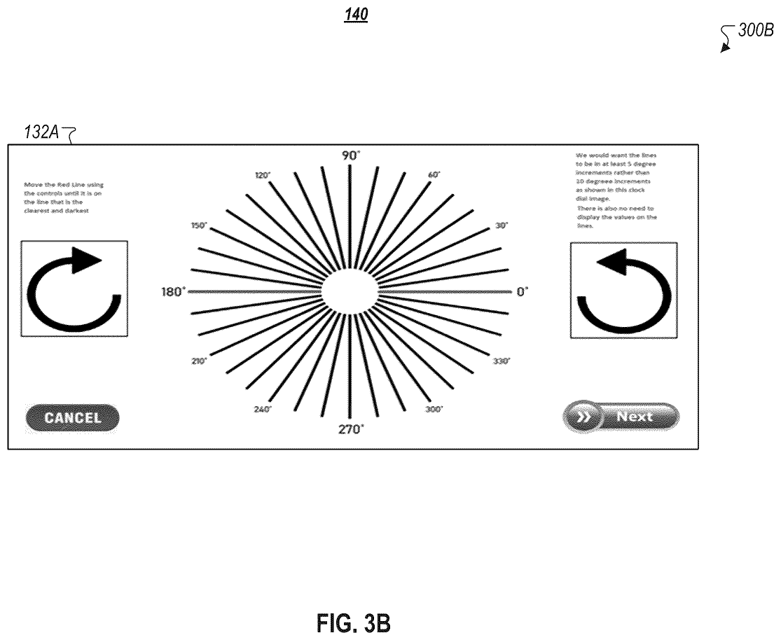

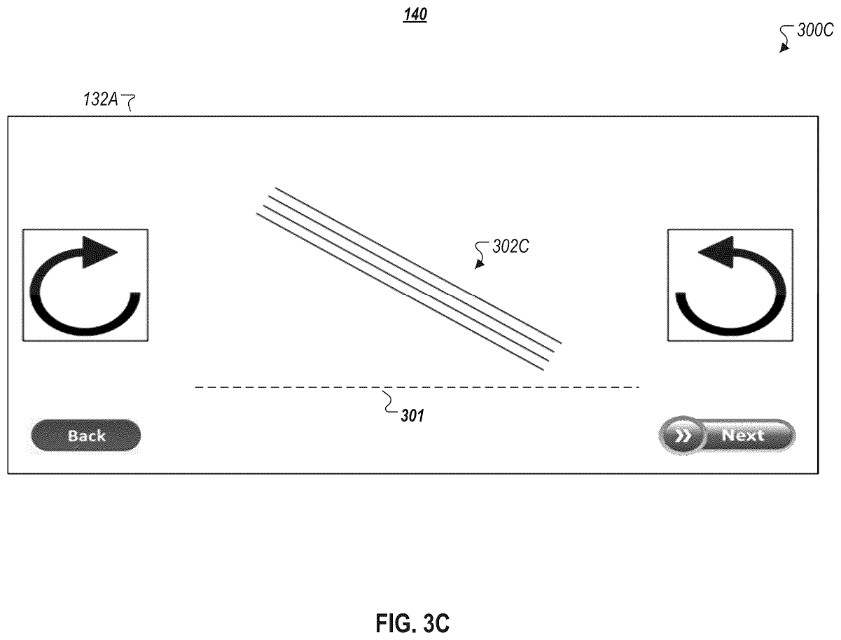

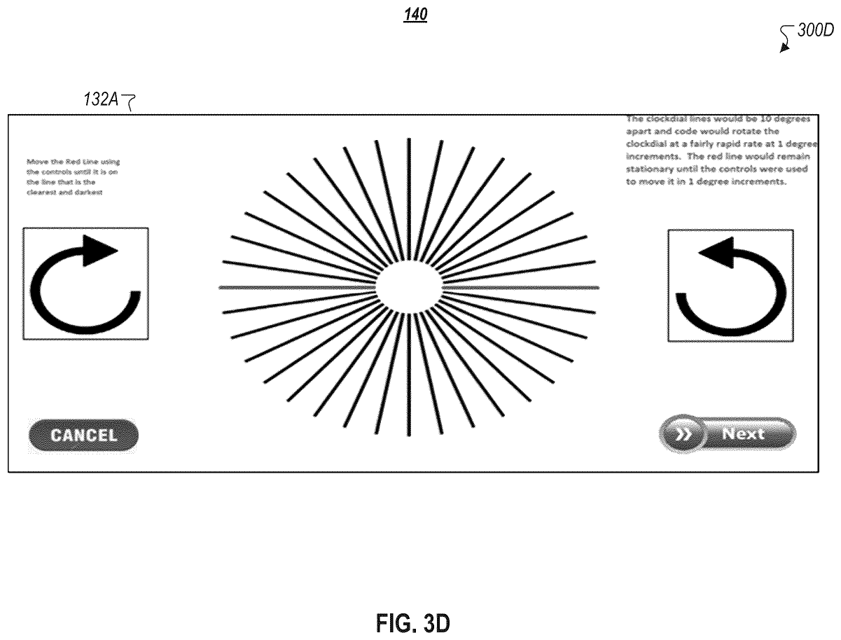

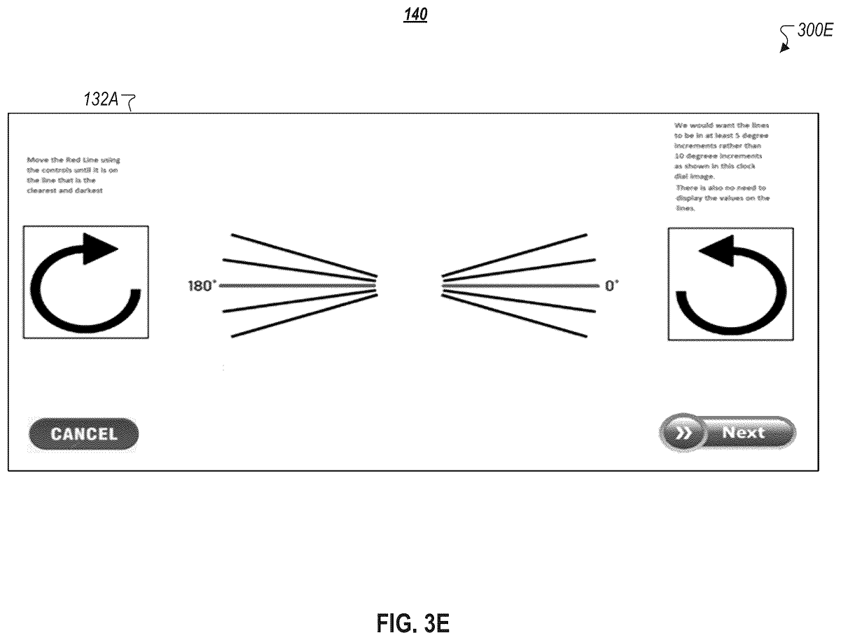

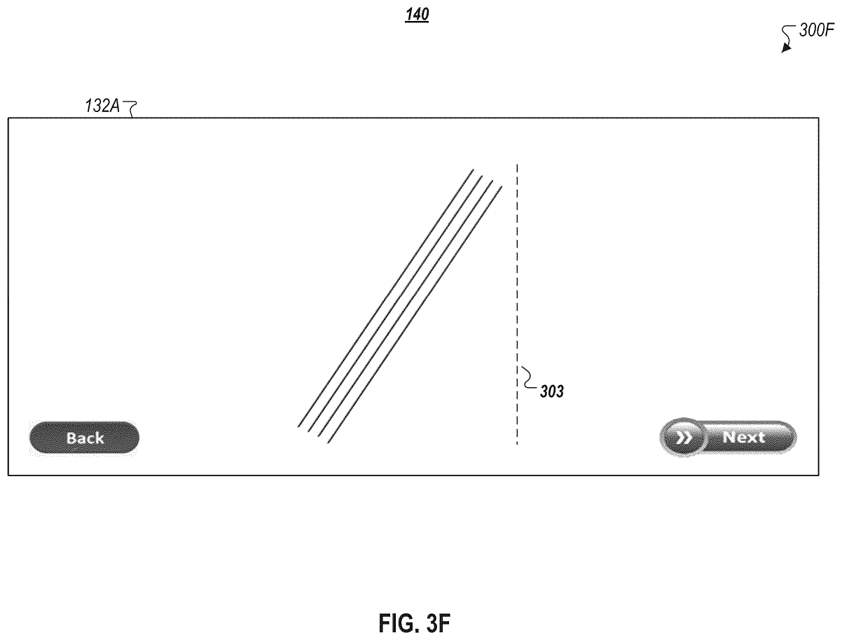

User device 130 and display 140 can be electronic devices of a user that are used to administer a remote vision examination. User device 130 and display 140 can be one or more of a smartphone, a tablet computing device, a laptop computing device, a desktop computing device, among others. User device 130 and display 140 permit a user to access a remote communication session with provider device 120 over network 105 and through which the user 102A can receive a remote vision examination. As described throughout, a user can utilize user devices 130 or provider device 120 to emulate the lens adjustments that would be performed, for example, during a traditional subjective refraction test. As discussed in more detail in relation to FIGS. 3A-3F the remote subjective refraction examination is accomplished by presenting a set of interfaces on display 140 and instructing the user 102A to perform actions to simulate the effect of trying on different trial lens. The user 102A performs an initial calibration operation on 140 so that data collected during the remote subjective refraction examination accurately reflects lens adjustments. The user 102A accesses the remote communication session through user device 130 though, in some instances, functionality described throughout for the user device 130 and display 140 can be combined into a single device that performs the operations of both.

In some implementations, the display 140 and the sensor 150 are physically and electrically integrated, i.e., embedded, into the user device 130, such as a smartphone, tablet, or laptop having an integrated camera. In other implementations, any one of the display 140 or sensor 150 can be embedded into the user device 130. In another example, the display 140 can be embedded into the sensor 150, and the user device 130 can be wirelessly or directly coupled to the embedded display 140 and sensor 150.

For example, in some implementations, the sensor 150 can be a webcam that is stand-alone from one or both of the display 140 and user device 130. In other implementations, the sensor 150 is embedded into the display 140, such as camera embedded into a stand-alone monitor or embedded into a laptop computer. In yet another implementation, each of the user device 130, display 140, and sensor 150 can be stand-alone device or coupled to one another either physically, wirelessly, or a combination of both.

FIG. 1B illustrates an example of a technique for facilitating a remote vision examination between the user 102A and the provider 102B. As shown in FIG. 1B, the technique involves a flow of information that is depicted in the figure as a set of steps relating to facilitating a remote vision examination.

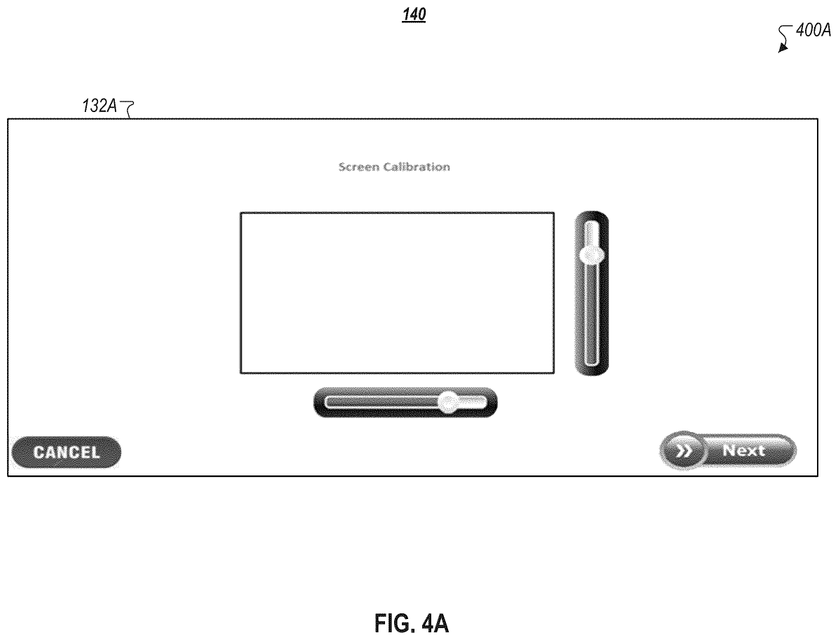

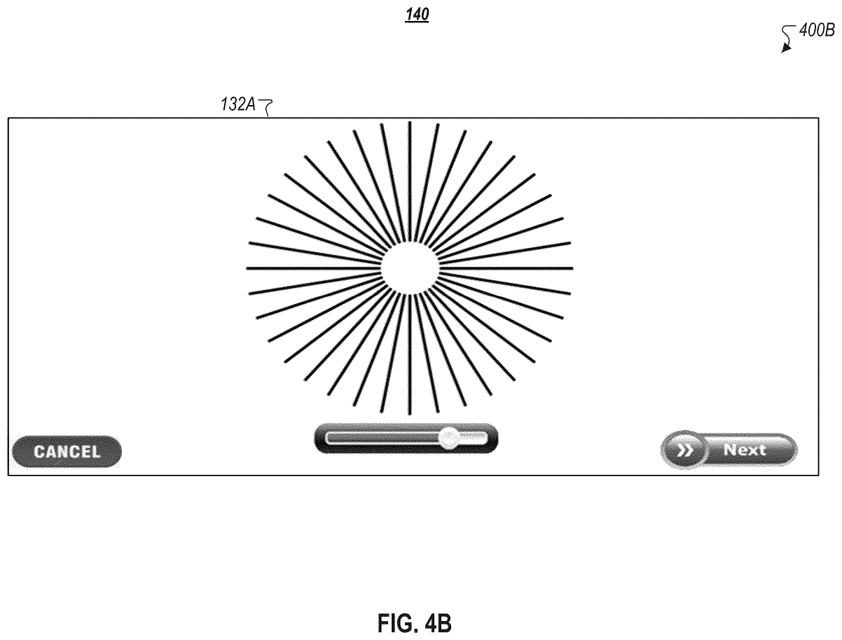

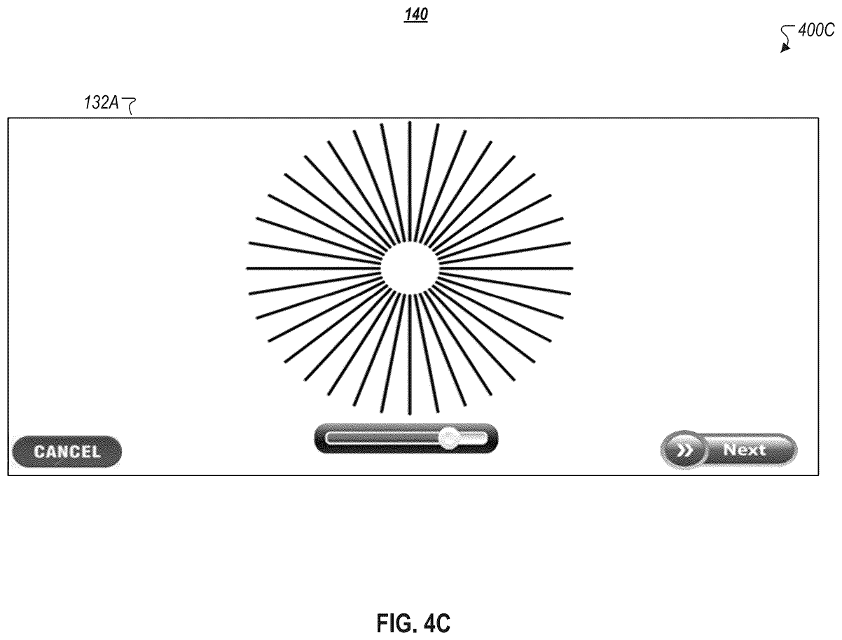

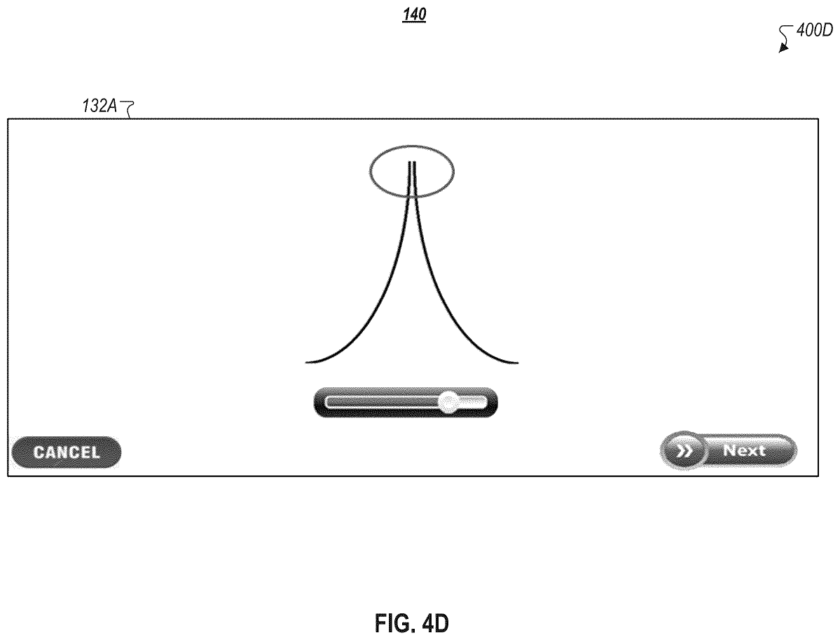

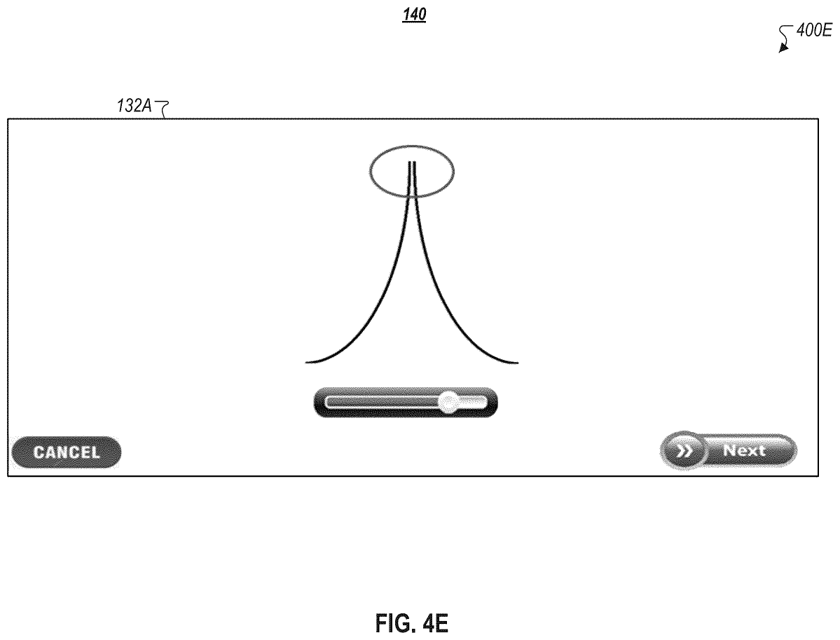

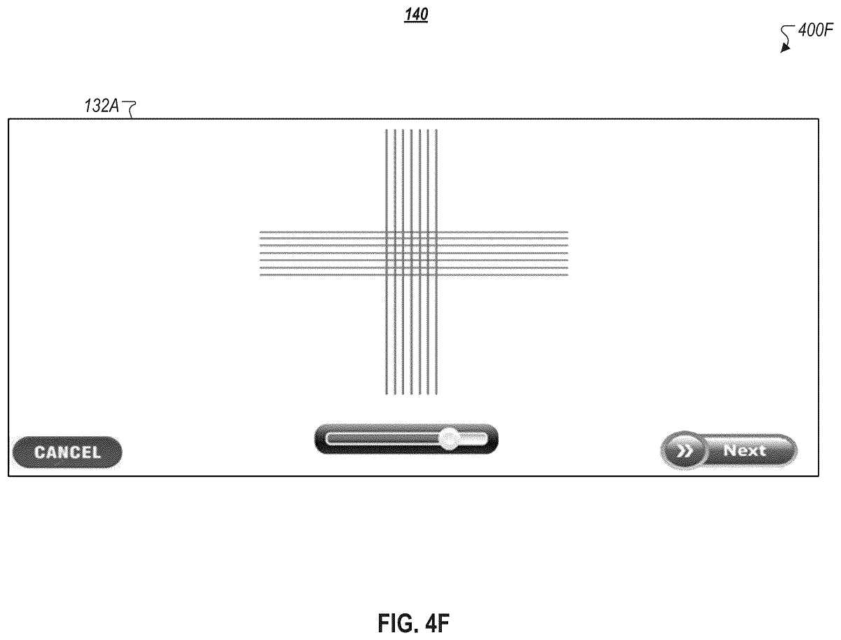

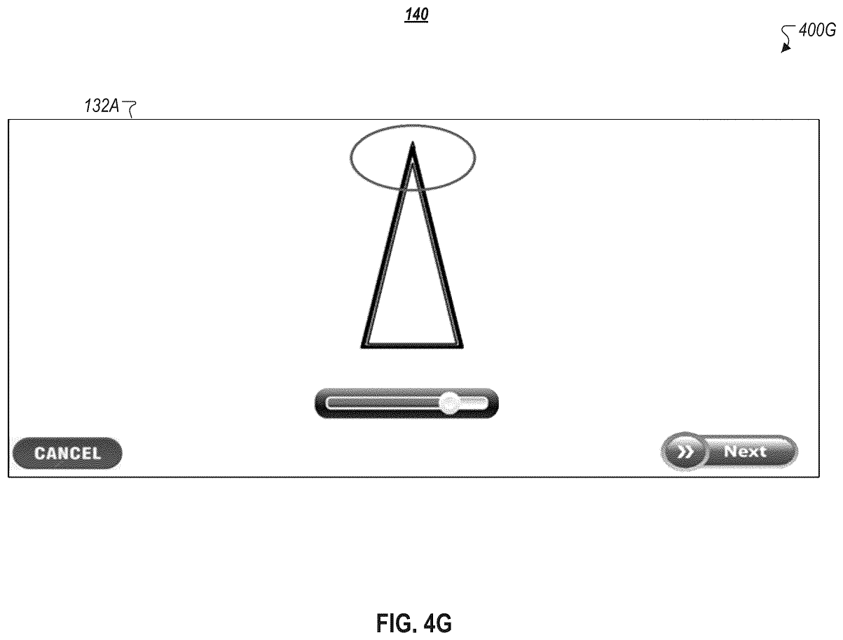

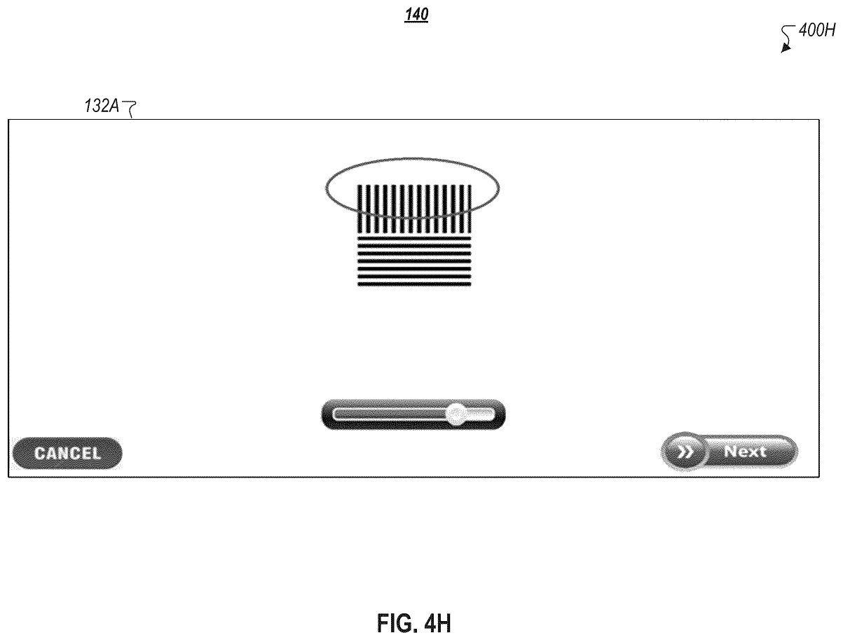

At step (1), user 102A accesses a calibration procedure through user device 130 that involves computing an egocentric distance 104 between the user 102A and display 140. In some implementations, user 102A accesses the calibration procedure through an application running on user device 130, such as a web-based application, a native software-based application (e.g., mobile application, desktop application). As shown in FIG. 1B, the user 102A accesses a calibration interface 132A to calibrate the display 140 for the remote vision examination. Other examples of calibration interfaces that may be used, for example, as shown in FIGS. 3A-3F and FIGS. 4A-4H in relation to two types of remote subjective refraction examinations.

At step (2), user device 130 sends calibration data 106 to server 110. The server 110 uses calibration data 106 to compute the egocentric distance 104. The egocentric distance 104 can be a distance between the sensor 150 and the user 102A. In some implementations, server 110 can implement one or more neural networks and/or use projective geometry to estimate the absolute depth of reference objects within a field of view (not shown in FIG. 1B) of the sensor 150. For example, neural network parameters are trained to regress to specific regions, or landmarks of a detected reference object, such as the face of the user 102A, or a reference object. The reference object is of a known dimension and is substantially isotropic, as explained in detail below. The reference object, a validation object, or a combination, can be used during user device calibration.

The server 110 can use a variety of processing techniques to compute the egocentric distance 104 and/or associated metrics in various implementations.

### Triangulation

In some implementations, the sensor 150 is an electro-optical sensor that captures a 2-D representation of a 3-D scene. Depth information is lost and can be distorted as the 3-D scene is projected onto the sensor 150 during image capture. Triangulation uses at least two known sides of similar triangles to estimate the third. The present disclosure uses the focal length of the sensor 150 and a known width between two points on a detected physical object. It follows that the distance to the subject, i.e., the user 102A, can be found using following equation:

$${D}_{subject} = \frac{f * {W}_{object}}{{W}_{pixel}}$$

where D is the subject-to-sensor 150 distance, f is the focal length of the sensor 150, W_{object} is the real width of the object, and Wₚᵢₓₑₗ is the width of the object in pixels on the display 140. Utilizing a neural network, in some implementations, after the focal length f is calculated, the object W_{object} can be re-identified and the new distance D can be derived from its new width in terms of number of pixels.

### Sensor Calibration

In another implementation, the focal length can be estimated, by for example, calculating one or more radial distortion coefficients. The focal length (f) and the one or more distortion coefficients, are stored by server 110 can be utilized to reduce distortion in acquired data from the sensor 150, and thereby improving the accuracy of distance (D) determination.

### Reference Objects

Server 110 determines one or more reference objects whose image(s) are captured by the sensor 150. For example, server 110 can use facial landmark detection techniques to obtain calibration data 106 from user 102A. In at least one example, a 468 landmark canonical face mesh is utilized, which provides for a selection of a wide variety of facial landmarks for user 102A. For example, an average diameter of either iris of a given user is between about 11.0 mm and about 12.5 mm. In another implementation, the facial landmark detection technique makes use of about 480 facial landmarks, with the additional landmarks include the horizontal and vertical axes of both irises, as captured by the sensor 150 and viewed on the display 140.

Given that the human iris is 11.7 mm with a standard deviation of roughly 0.5 mm, the user's iris can be used to estimate distances between other facial landmarks on the canonical face mesh. A neural network trained to estimate the 468 points of the canonical face mesh model and iris points can leverage this knowledge, using the iris' assumed width to estimate the Euclidean distances between other facial landmarks. During a biometric calibration, statistics can be gathered and stored for later use. In the event where the user moves beyond the point at which the iris' width is accurately captured (e.g., greater than a threshold distance), the facial landmark detection technique captures other landmarks on the user's face. In some implementations, facial landmark detection techniques identify additional facial landmarks when the user's distance from the sensor is greater than the threshold distance. In another implementation, an estimated iris measurement is used to calculate the distance between other facial landmarks that are identifiable at distances greater than the threshold distance. A neural network, trained on a dataset configured to identify the canonical face mesh, can identify one or more biological reference objects. The threshold distance is between about 2 m and 6 m, such as about 3 m, or about 4 m, or about 5 m.

In some implementations, a validation object of standardized dimensions may be used to further refine the measurements obtained from the reference object. Validation objects, such as identification or credit cards can be used to cross validate the biometric measurement to assure that the detected width is within an acceptable range. In some examples, the validation object can be an identification card, such as a license, credit card, or membership card.

### Display Calibration

In some implementations, server 110 determines a pixel density of display 140. Pixel density is determined by adjusting images projected on display 140 to fit the size of a standardized reference object, such as a credit card. Once the user 102A is satisfied that the boundaries on their display represent the boundaries of the reference object, the corresponding dimension of the reference object can divide the number of whole pixels contained in the vertical or horizontal dimensions, producing the number of pixels per unit of measure. This scalar value is used to appropriately size optotypes on the Snellen chart in a dynamic fashion, such that each optotype subtends an angle of 5 arcminutes at the user's 120A egocentric distance from the sensor 150.

Snellen charts typically utilize at least two or more criteria, such as a measurement of minimum separable acuity and the measurement of minimum recognizable acuity. Optotypes (letter indicators on a Snellen chart) are constructed such that each letter indicator's distinguishing features have a minimum angle of resolution of 1 minute of arc. Digitally produced optotypes can be dynamically increased or decreased in size, in response to the user's distance to display 10. For example, the digitally produced optotypes increase in size as the user's distance increases from the sensor. Accordingly, pixel density of display 140 can be accounted for and the optotype's minimum angle of resolution. Advantageously, the digitally produced optotypes minimize the operational constraints on user 102A.

Standard vision is defined as the user's ability to recognize the optotypes subtending at an angle of 5 minutes of arc and recognizing that each critical detail subtends an angle of 1 minute of arc. Using Snellen's fraction, it is possible to define visual acuity utilizing the following equation.

$$Snellen ' s fraction = \frac{Distance to Snellen Chart}{Distance at which smallest optotypes subtend an angle of {5}^{′} arc}$$

The height of each optotype, i.e. letter, is represented by the following equation:

$$Height of Letter = tan ( \frac{{5}^{′}}{60} ) ( distance to Snellen Chart )$$

While the height of the optotype is known, the dimensions and resolution of the display 140 may be unknown. The validation object can be used to determine the pixel density. The user 102A proceeds by holding the validation object proximate the screen and fitting the circumference of the validation object within the perimeter of a sizing box, e.g., calibration box 302A discussed below, that enables determination of the pixel density of the display 140. For example, a typical credit card is about 85.6 mm×53.98 mm×0.76 mm.

At step (3), server 110 evaluates the calibration data 106 in relation to a set of criteria 112B. The criteria 112B generally specifies requirements to ensure a sufficient accuracy for examination data generated during a remote vision examination. Examples of criteria 112B include a minimum internet connection speed, a physical positioning between one or more of user device 130, display 140, and sensor 150, sensor parameters of sensor 150, a resolution of display 140, among others, lighting conditions for a location of user 102A. In some implementations, the criteria 112B specifies a connection speed of the network 105 with an upload and download speed of at least about 50 Mbps. This ensures minimal or no latency while the remote vision examination is administered by provider 102B over a wide area network. Alternatively, or additionally, the criteria 112B specifies a minimum luminosity of about 500 lux for the user's location. This ensures that a user can view the information provided on display 140 and provide correct responses and/or collected examination data appropriately reflects visual acuity of the user 102A.

At step (4), server 110 determines that criteria associated with the calibration data 106 have been satisfied and confirms the calibration to user device 130. Server 110 may make this determination based on comparing values specified by calibration data 106 and values specified in criteria 112B. For example, server 110 determines that a minimum luminosity value of 500 lux specified in criteria 112B is satisfied based on calibration data 106 indicating a measured luminosity value of 600 lux. In other instances, server 110 makes the determination based on evaluating multiple values and/or conditions specified by the server 110. For example, if server 110 can determine that the criteria 112B are satisfied based on information specified by calibration data 106 having values that satisfy four of six total criterion. In some other instances, the determination is not strictly based on values, but may further be based on holistically analyzing various types of information specified by calibration data 106. For example, if the Internet speed of user device 130 is below a specified threshold, server 110 may nonetheless determine satisfaction of criteria 112B if display 104 has a sufficiently high resolution (e.g., 4K resolution, or 3840 pixels by 2160 pixels) and/or if sensor 150 uses a sufficiently high video capture technique.

At steps (5A) and (5B), server 110 sends vision examination instructions to user device 130 and provider device 120, respectively, based on the confirmed calibration. The instructions provided to provider 120 and user device 130 enable the device to establish a synchronous or asynchronous connection over network 105. At step (6), server 110 facilitates establishing a connection between user device 130 and provider device 120 for administering a remote vision examination. The remote connection permits provider 102B to communicate with user 102A via provider device 120 and user device 130, respectively. The connection may be a video conference or an audio conference, and further, may be synchronous or asynchronous depending on the constraints and/or desired requirements for a remote vision examination.

In some implementations, the connection between user device 130 and provider device 120 is a synchronous communication session during which the provider 102B and the user 102A are able to communicate in real-time (or substantially in real-time). In other implementations, the connection between user device 130 and provider device 120 is an asynchronous communication session in which the user 102A initially performs a predetermined set of actions to generate preliminary examination data and, at a later time point, the provider 102B evaluates the examination data to determine a best corrected visual acuity. In some instances where the preliminary examination data is insufficient to determine a best corrected visual acuity, the provider 102B may subsequently request a follow-up communication to obtain additional examination data. For example, the provider 102B may request the user 102A to provide additional examination data (e.g., additional configurations of the 140 and corresponding vision feedback) or a follow-up synchronous communication session (e.g., a communication session during which the provider 102B conducts a follow-up subjective refraction examination in real-time).

At steps (7A) and (7B), provider 102B administers the remote vision examination as the user interacts with examination interface 132B. Provider 102B administers the remote vision examination by conducting similar types of evaluations and/or tests that may be performed by an in-person vision examination. For example, provider 102B may perform actions that emulate a subjective refraction examination using trial lenses and is used to determine visual acuity. User 102A can be instructed to move to specified distances from an eye chart at which the user's unaided visual acuity can be measured for each eye (and then binocularly).

At step (8), upon completion of the remote vision examination, provider device 120 sends server 110 examination data for processing. After receipt from the provider 102B, server 110 transmits the examination data to the user device 130. The examination data may be presented on the display 140 via examination interface 132B. In one example, the examination data may be information representing a Snell's chart.

Examination data is generated based on actions performed by the user 102A during the remote subject refraction examination (e.g., adjusting the distance from display 140 and corresponding visual acuity measurements). Examination results are the accumulation of examination data throughout the remote communication session. In some implementations, user 102A may provide their current prescription to provider 102B through network 105. The current prescription can be stored on server 110, or locally on user device 130. As such, the current prescription can be used as a basis for determining the calibration criteria. The examination results are stored on the server 110.

As noted above, examination data can include information collected during a remote vision examination, test results, configuration instructions, a user identification, among other information related to the user device 130 and/or provider device 120. Information related to examination data is stored at the server 110 can originate from either the user 102A, the provider 102B, the server 110, or a combination thereof.

At steps (9A) and (9B), server 110 processes examination data for output. At step (9A), server 110 stores the processed examination data as examination results 114B. Further server 110 provides examination for output to user device 130 (not shown in FIG. 1B) so that user 102A can access examination results and/or prescription information via prescription interface 132C. The stored information can relate to, for example, visual acuity, refractive errors, eyewear prescription (e.g., best corrected visual acuity), and/or examination results.

For example, a computer-generated image of an astigmatic charts 300B (shown in FIG. 3B), 300D (shown in FIG. 3D), and 300E (shown in FIG. 3E) include a clock dial and visual targets that can be used to refine axis. Chart 300B includes a clock dial that can be used to do an initial axis check. A user is instructed to move the redline using input control until it is on the line that is the clearest and darkest.

As another example, axis determination charts 300C (shown in FIG. 3C) and 300F (shown in FIG. 3F) include lines oriented at approximately 90 degrees away from a first axis 301. Parallel lines 302C can be used as a method to refine the axis after the clock dial (shown in chart 300B) has been used. A visual element (“E”) can also be used that is rotated on the screen with the three parallel lines of the “E” being in parallel with the first axis 301. As to chart 300F, after the axis of the first meridian is found, parallel lines could be displayed on a second axis 303 that is exactly 90 degrees from the first axis 301 to find the focal point of the second meridian if astigmatism is present. The “E” can also be used that is rotated on the screen with the 3 lines of the E on the second meridian axis.

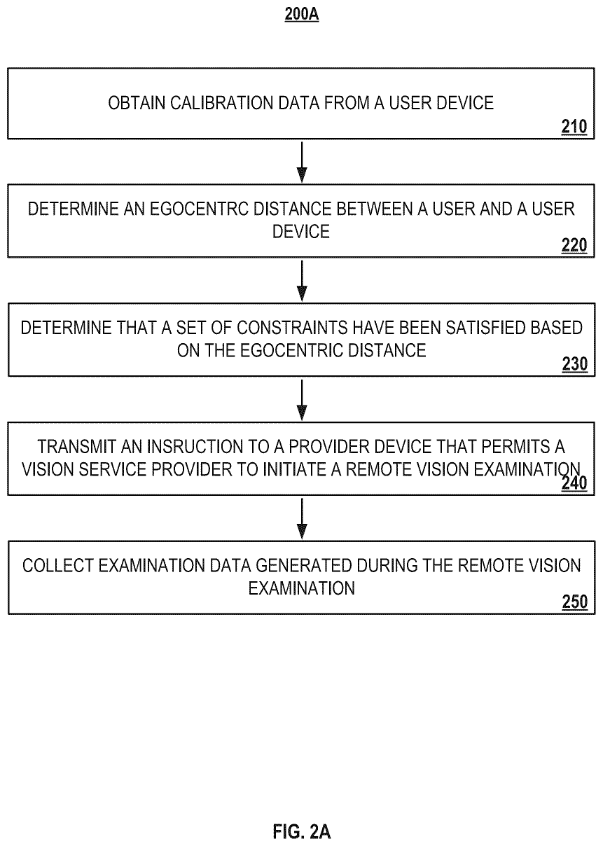

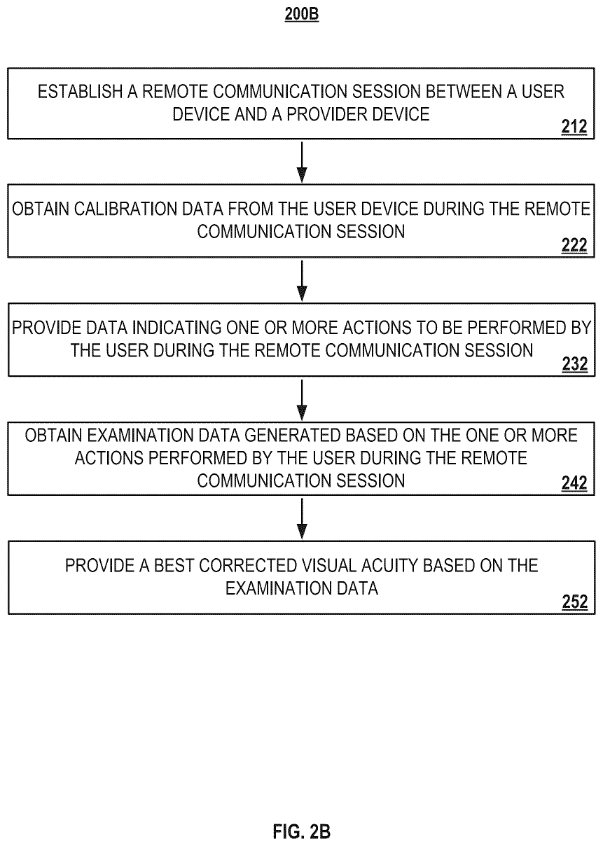

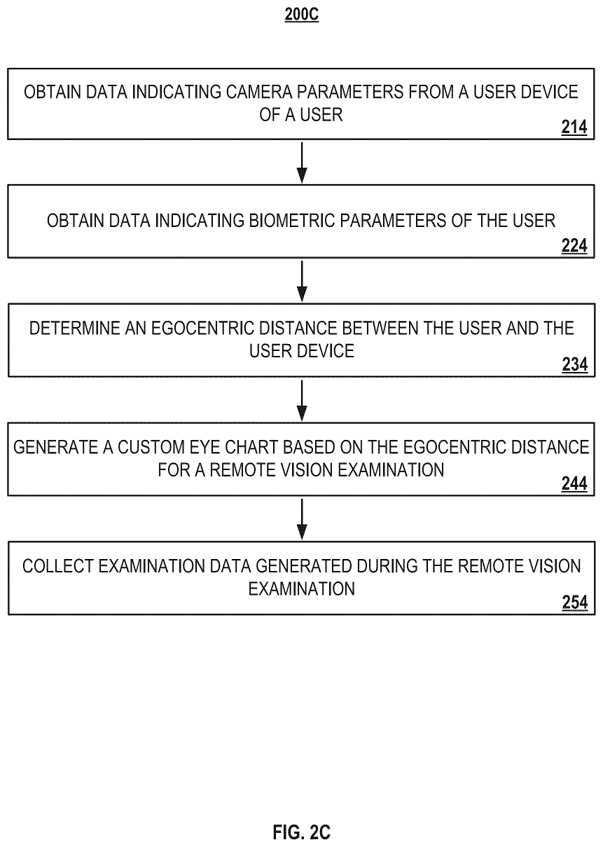

FIG. 2A illustrates an example of a process 200A for administering remote vision examinations using automated egocentric distance calibration. Briefly, the process 200A includes the operations of obtaining calibration data from the user device (210), determining an egocentric distance between a user and one or more user devices (220), determining that a set of criteria have been satisfied based on the egocentric distance (230), transmitting an instruction to a provider device that permits a vision service provider to initiate a remote vision examination (240), establishing a communication session between the one or more user devices and the provider device (250), collecting examination data generated during the remote vision examination (260), and transmitting the examination data in a manner that permits the vision service provider to at least evaluate the examination data (270).

The process 200A includes the operation of obtaining calibration data from the user device (210). For example, the server 110 may receive calibration data relating to an eye chart from user device 130 during the remote communication session. Calibration can be performed using display 140 and, in some implementations, an associated image capture device (e.g., webcam). For example, interface 300A (shown in FIG. 3A) can be displayed on display 140 and a user may position the card to match a corresponding interface element displayed on interface 300A.

For example, server 110 receives calibration data 106 from the user device 130, as shown in FIG. 1B. Calibration data 106 relates to an eye chart shown on, for example, calibration interface 132A. Calibration can be performed using display 140 and an associated sensor 150, (e.g., an image capture device, such as a webcam, digital camera, computer-embedded camera, and the like). For example, an interface 300A (shown in FIG. 3A) can be displayed on display 140 and a user may position the card to match a corresponding interface element displayed on interface 300A. In some implementations, the sensor 150 is a standard high-definition imaging sensor having a resolution of 1080p or greater. The calibration may utilize a reference object, a validation object, or combination.

The process 200A includes the operation of determining an egocentric distance between a user and one or more user devices (220). The server 110 can use calibration data 106 to compute the egocentric distance 104. The egocentric distance 104 can be a distance between the sensor 150 and the user 102A. In some implementations, server 110 can implement one or more of a neural networks and projective geometry to estimate the absolute depth of objects within a field of view of the sensor 150. For example, neural network parameters are trained to regress to specific regions, or landmarks of a detected object, such as the face of the user 102A, or a reference object. The reference object is of a known dimension and is substantially isotropic, as explained in detail below. The reference object, a validation object, or a combination, can be used during user device calibration.

The process 200A includes the operation of determining that a set of criteria have been satisfied based on the egocentric distance (230). Server 110 may make this determination based on comparing values specified by calibration data 106 and values specified in criteria 112B. For example, server 110 determines that a minimum luminosity value of 500 lux specified in criteria 112B is satisfied based on calibration data 106 indicating a measured luminosity value of 600 lux. In other instances, server 110 makes the determination based on evaluating multiple values and/or conditions specified by the server 110. For example, if server 110 can determine that the criteria 112B are satisfied based on information specified by calibration data 106 having values that satisfy four of six total criterion. In some other instances, the determination is not strictly based on values, but may further be based on holistically analyzing various types of information specified by calibration data 106. For example, if the Internet speed of user device 130 is below a specified threshold, server 110 may nonetheless determine satisfaction of criteria 112B if display 104 has a sufficiently high resolution (e.g., 4K resolution, or 3840 pixels by 2160 pixels) and/or if sensor 150 uses a sufficiently high video capture technique.

The process 200A includes the operation of transmitting an instruction to a provider device that permits a vision service provider to initiate a remote vision examination (240).

The process 200A includes the operation of establishing a communication session between the one or more user devices and the provider device (250). For example, server 110 establishes a communication session between user device 130 and provider device 120. In some implementations, the remote communication session is a synchronous communication session (e.g., audio conference, video conference) during which a provider and a user are able to communicate in real-time (or substantially in real-time). In such implementations, the provider instructs the user to make certain adjustments to user device 130 and display 140 to emulate the process of trying different trial lenses for vision correction. For example, as discussed in reference to FIGS. 3A-3G, the user can be asked to access different user interfaces on display 140 and asked to adjust, for instance, the distance between him/her and the display 140 and then provide feedback relating to clarity of elements displayed on the user interfaces. In other implementations, the remote communication sessions is an asynchronous communication session in which a user initially performs a predetermined set of actions to generate preliminary examination data and, at a later time point, a provider evaluates the examination data to determine a best corrected visual acuity. In some instances where the preliminary examination data is insufficient to determine a best corrected visual acuity, the provider may subsequently request a follow-up communication to obtain additional examination data. For example, the provider may request the user to provide additional examination data (e.g., additional configurations of the device 140 and corresponding vision feedback) or a follow-up synchronous communication session (e.g., a communication session during which the provider conducts a follow-up subjective refraction examination in real-time).

The process 200A includes the operation of collecting examination data generated during the remote vision examination (260). For example, server 110 may receive examination data from provider device 120 and generated based on actions performed by the user during the remote vision examination (e.g., adjusting the distance from display 140 and corresponding visual acuity measurements).

The process 200A includes the operation of transmitting the examination data in a manner that permits the vision service provider to at least evaluate the examination data (270). For example, server 110 can provide examination data collected from user device 130 during the remote vision examination to the provider device 120. The provider 102B can use the examination data to determine a best corrected visual acuity, which is presented to the user 102A on user device 130 via prescription interface 132C.

In various implementations, the connection between user device 130 and provider device 120 can be established at different time points in relation to generation of calibration data 106 at user device 130 and/or computation of the egocentric distance 104. For example, in some implementations, the connection is established prior to calibration, which allows user 102A to communicate with provider 102B before or during calibration. For example, newer users may benefit from the interaction with the provider 102B to receive direct assistance with setting up user device 130 for a remote vision examination. In other implementations, the connection is established after a calibration procedure, which helps reduce bandwidth required by the network 105 by enabling additional users with the bandwidth that would otherwise be utilized to connect the user device 130 with provider device 120. Such implementations provide additional advantages in that they reduce the total time the provider 102B needs to dedicate to administering a remote vision examination.

FIG. 2B illustrates an example of a process 200B for administering a remote vision examination using manual egocentric distance calibration. Briefly, the process 200B includes the operations of establishing a remote communication session between a user device and a provider device (212), obtaining calibration data from the user device during the remote communication session (222), providing data indicating one or more actions to be performed by the user during the remote communication session (232), obtaining examination data generated based on the one or more actions performed by the user during the remote communication session (242), and providing a best corrected visual acuity for the user based on the examination data (252).

In more detail, the process 200B includes the operations of establishing a remote communication session between a user device and a provider device (212). For example, server 110 establishes a communication session between user device 130 and provider device 120. In some implementations, the remote communication session is a synchronous communication session (e.g., audio conference, video conference) during which a provider and a user are able to communicate in real-time (or substantially in real-time). In such implementations, the provider instructs the user to make certain adjustments to user device 130 and display 140 to emulate the process of trying different trial lenses for vision correction. For example, as discussed in reference to FIGS. 3A-3G, the user can be asked to access different user interfaces on display 140 and asked to adjust, for instance, the distance between him/her and the display 140 and then provide feedback relating to clarity of elements displayed on the user interfaces. In other implementations, the remote communication sessions is an asynchronous communication session in which a user initially performs a predetermined set of actions to generate preliminary examination data and, at a later time point, a provider evaluates the examination data to determine a best corrected visual acuity. In some instances where the preliminary examination data is insufficient to determine a best corrected visual acuity, the provider may subsequently request a follow-up communication to obtain additional examination data. For example, the provider may request the user to provide additional examination data (e.g., additional configurations of the device 140 and corresponding vision feedback) or a follow-up synchronous communication session (e.g., a communication session during which the provider conducts a follow-up subjective refraction examination in real-time).

The process 200B includes the operation of obtaining calibration data from the user device during the remote communication session (222). For example, the server 110 may receive calibration data relating to an eye chart from user device 130 during the remote communication session. Calibration can be performed using display 140 and an associated image capture device (e.g., webcam). For example, interface 300A (shown in FIG. 3A) can be displayed on display 140 and a user may position the card to match a corresponding interface element displayed on interface 300A.

The process 200B includes the operation of providing data indicating one or more actions to be performed by the user during the remote communication session (232). For example, the server 110 can relay data indicating instructions from the provider for the user to perform actions relating to display 140. As discussed throughout, the actions performed by the user can be used to emulate a subjective refraction examination using trial lenses and is used to determine visual acuity. For example, a user can be instructed to move to specified distances from an eye chart at which the user's unaided visual acuity can be measured for each eye (and then binocularly).

The process 200B includes the operation of obtaining examination data generated based on the one or more actions performed by the user during the remote communication session (242). Examination data is generated based on actions performed by the user during the remote subject refraction examination (e.g., adjusting the distance from display 140 and corresponding visual acuity measurements).

For example, an astigmatic charts 300B (shown in FIG. 3B), 300D (shown in FIG. 3D), and 300E (shown in FIG. 3E) include a clock dial and visual targets that can be used to refine axis. Chart 300B includes a clock dial that can be used to do an initial axis check. A user is instructed to move the redline using input control until it is on the line that is the clearest and darkest.

As another example, axis determination charts 300C (shown in FIG. 3C) and 300F (shown in FIG. 3F) include lines oriented at approximately 90 degrees away from a first axis. Parallel lines can be used as a method to refine the axis after the clock dial (shown in chart 300B) has been used. An visual element (“E”) can also be used that is rotated on the screen with the three lines of the “E” on the same axis. As to chart 300F, after the axis of the first meridian is found, parallel lines could be displayed on an axis that is exactly 90 degrees from the first axis to find the focal point of the second meridian if astigmatism is present. The “E” can also be used that is rotated on the screen with the 3 lines of the E on the second meridian axis.

The process 200B includes the operation of providing a best corrected visual acuity for the user based on the examination data (252). For example, the provider can use the examination data generated during the remote subjective refraction examination to determine a best corrected visual acuity. FIG. 2C illustrates an example of a process 200C for calibrating user devices for a remote vision examination.

FIG. 2C illustrates an example of a process 200C for calibrating user devices for a remote vision examination. Briefly, the process 200C includes the operations of obtaining data indicating camera parameters from a user device of a user (214), obtaining data indicating biometric parameters of the user (224), determining an egocentric distance between the user and the user device (234), generating a custom eye chart based on the egocentric distance for a remote vision examination (244), and collecting examination data generated during the remote vision examination (254).

In more detail, the process 200C includes the operation of obtaining data indicating camera parameters from a user device of a user (214). Server 110 obtains data from user device 130 to perform screen calibration. As described above, examples of camera parameters includes a focal length of a camera, one or more distortion coefficients associated with radial distortion of a lens of the camera, camera configuration information (e.g., shutter speed, aperture, ISO values), among others. In some implementations, camera parameters are used to identify depth information, which can an angle of 5 minutes of arc then be used to determine the egocentric distance 104.

The process 200C includes the operation of obtaining data indicating biometric parameters of the user (224). Server 110 obtains data from user device 130 to perform biometric calibration. As described above, examples of biometric parameters include facial landmarks, iris indicators (e.g., iris diameter, iris distance, etc.), among others. In some implementations, the biometric parameters are used to generate a canonical face mesh model in performing a biometric calibration prior to a remote vision examination. During a biometric calibration, statistics can be gathered and stored for later use. In the event where the user moves beyond the point at which the iris' width is accurately captured (e.g., greater than a threshold distance), the facial landmark detection technique captures other landmarks on the user's face. In some implementations, facial landmark detection techniques identify additional facial landmarks when the user's distance from the sensor is greater than the threshold distance. In another implementation, an estimated iris measurement is used to calculate the distance between other facial landmarks that are identifiable at distances greater than the threshold distance. A neural network, trained on a dataset configured to identify the canonical face mesh, can identify one or more biological reference objects. The threshold distance is between about 2 m and 6 m, such as about 3 m, or about 4 m, or about 5 m.

The process 200C includes the operation of determining an egocentric distance between the user and the user device (234). Server 110 can use calibration data 106 to compute the egocentric distance 104. As discussed above, the egocentric distance 104 can be a distance between the sensor 150 and the user 102A. In some implementations, server 110 can implement one or more of a neural networks and projective geometry to estimate the absolute depth of objects within a field of view of the sensor 150. For example, neural network parameters trained to regress to specific regions, or landmarks of a detected object, such as the face of the user 102A, or a reference object. The reference object is of a known dimension and is substantially isotropic, as explained in detail below. The reference object, a validation object, or a combination, can be used during user device calibration.

The process 200C includes the operation of generating a custom eye chart based on the egocentric distance for a remote vision examination (244). Server 110 can generate an eye chart for display on examination interface 132B during the remote vision examination. The generated eye chart is customized based on camera parameters and biometric parameters obtained in operations 214 and 224, respectively. For example, server 110 can adjust the size of letter indicators of the eye chart (shown on display 140) based on the egocentric distance 140 to increase the likelihood of performing an accurate vision assessment. In other examples, the server 110 may adjust other aspects of the eye chart, such as the number of rows to include in an eye chart, spacing between individual letter indicators, the number of letter indicators to include in an eye chart, among others. The eye chart can also be customized based on biometric parameters obtained from user 130. As described throughout, calibration enables display 140 to display an eye chart using the proper size of letter indictors for purpose of checking the visual acuity.

The process 200C includes the operation of collecting examination data generated during the remote vision examination (254). For example, server 110 establishes a communication session between user device 130 and provider device 120. In some implementations, the remote communication session is a synchronous communication session (e.g., audio conference, video conference) during which a provider and a user are able to communicate in real-time (or substantially in real-time). In such implementations, the provider instructs the user to make certain adjustments to user device 130 and display 140 to emulate the process of trying different trial lenses for vision correction. For example, as discussed in reference to FIGS. 3A-3G, the user can be asked to access different user interfaces on display 140 and asked to adjust, for instance, the distance between him/her and the display 140 and then provide feedback relating to clarity of elements displayed on the user interfaces. In other implementations, the remote communication sessions is an asynchronous communication session in which a user initially performs a predetermined set of actions to generate preliminary examination data and, at a later time point, a provider evaluates the examination data to determine a best corrected visual acuity. In some instances where the preliminary examination data is insufficient to determine a best corrected visual acuity, the provider may subsequently request a follow-up communication to obtain additional examination data. For example, the provider may request the user to provide additional examination data (e.g., additional configurations of the device 140 and corresponding vision feedback) or a follow-up synchronous communication session (e.g., a communication session during which the provider conducts a follow-up subjective refraction examination in real-time).

FIGS. 3A-3F illustrate a series of computer-generated interfaces 300A-300F shown on the display 140 to be viewed by the user during one exemplary remote subjective refraction examination. In the example shown in FIGS. 3A-3F, the examination technique can be used for nearsighted patents. Display 140 initially shows an interface 300A, which includes calibration interface 132A, as depicted in FIG. 3A. The user 102A receives instructions to hold a validation object (e.g., driver's license, credit card, or some other card-sized document) at a first distance (e.g., one foot) from the center of display 140. An image of the validation object is captured and measured, and measurement in pixels is stored for the user 102A and related to the first distance. The interface 300A includes a calibration box 302A that is substantially in the shape of a parallelogram, e.g., a rectangle.

The calibration box 302A includes a vertical adjustment 304A that adjust a height of the calibration box 302A in a lengthwise direction of the user device 130. A horizontal adjustment 306A changes the width of the calibration box 302A in width-wise direction of the user device 130.

The user 102A receives a prompt at the user device 130 instructing the user 102A to hold the validation object at a second distance (e.g., 10 feet) from the center of display 140. An image of the card will be captured and measured, and that measurement in pixels will be stored for that user and related to the second distance. A screen calibration form is then displayed on display 140 and the user 102A is provided instructions, through the user device 130, to adjust the vertical and horizontal dimensions of the calibration box 302A until the dimension of the calibration box 302A matches the size of the validation object. Calibration enables the display 140 to display an eye chart using the proper size of letters for purpose of checking the visual acuity.

For calibration of user device 130, the size of the validation object at ten feet should be roughly the same as the size of the validation object at one foot divided by eight unless the image capture device does not maintain the same image-size ratio as the image gets closer or further from the image capture device (as may be the case with a fisheye camera lens). Any difference in in the calibration ratio found at ten feet and 1 foot can be checked using the following formulas:

CalDifference=ABS((10FootCalSize/10.00)−1FootCalSize) Equation 1.

If CalDifference is less than a threshold value, then the egocentric distance 104 can be computed using linear interpolation by using the two validation object sizes and calibration distances as a point on a line. If CalDifference is greater than the threshold value, then the image capture device may have a fisheye camera lens that may not be fully suitable for distance computations. One way the image capture device may work with the fisheye camera lens would be to collect a third distance calibration at a distance of, for instance, three or four feet, and use a different interpolation method such as spline interpolation.

As an example, the user 102A is initially instructed to move to a distance (e.g., least 10 feet) and use an eye chart to check the user's unaided visual acuity individually on each eye and then binocularly. For this example, the egocentric distance 104 in the first meridian is computed using the formula:

1stMeridianMMDistance=Linear(1stMeridianTargetSize,1FootCalSize, 1.00,10FootCalSize,10.00)×304.8 Equation 2.

This formula uses the conversion factor of 1-foot is equal to 304.8-mm. The egocentric distance 104 in the second meridian is computing using the formula:

2ndMeridianMMDistance=Linear(2ndMeridianTargetSize,1FootCalSize, 1.00,10FootCalSize,10.00)×304.8 Equation 3.

The user 102A receives a prompt at the user device 130 instructing the user 102A to cover the eye not being tested and to keep it covered through the remaining tests for the eye subject to testing. The user 102A is then instructed to move at least ten feet away from the display 140 and receives instructions to move closer slowly at certain increments. This process is repeated using the interface shown in FIG. 3B until one or more of the spokes on a clock dial looks its sharpest compared to the spoke that is exactly 90 degrees from that one. The user 102A needs to find the furthest distance that makes the best spokes clear and no closer to control accommodation. If all spokes are equally clear, the data indicating the egocentric distance 104 from the display 140 is stored locally on the user device 130 or remotely on the server 110, and a best corrected visual acuity is generated based on this information.

During the examination technique referenced above, one or more axis determination charts are used to fine tune the axis if needed to obtain maximum sharpness at that meridian. Data indicating the axis is stored locally on the user device 130 or remotely on the server 110. An egocentric distance 104 is computed by providing instructions through the user device 130 to the user 102A to again position the document with their eyes, and then click “Next” (depicted in the lower right side of FIG. 3B). An image of the document is captured and measured in pixels. This data is stored and used to compute the exact distance from the user's eyes to the image capture device using certain formulas.

A string of visual elements (e.g., string of “E” letters) is displayed on the screen along the axis, as discussed above. The visual elements are made progressively smaller until the user 102A can no longer tell that the visual element is included along this line. The visual acuity is represented by the size of the visual element considering the screen calibration values.

Parallel lines axis determination charts 300B (shown in FIG. 3B) and 300F (shown in FIG. 3F) have lines oriented exactly 90 degrees away from a first axis that was found in the previous operations discussed above. The user 102A is instructed to move closer to the display 140 slowly until the parallel lines become as clear without moving any closer than necessary to make them clear to control accommodation. The egocentric distance 104 is computed by prompting the calibration interface 132 to instruct the user 102A to position the reference object on a virtual plane that is even with their eyes. An image of the validation object is captured and measured in pixels, and the pixel value is stored and used to compute the exact distance from the user's eyes to the image capture device. The techniques discussed above can be repeated for the user's second eye to enable binocular visual acuity measurement.

In some implementations, formulas are used to compute the prescription a user needs to see clearly at “infinity” during the remote subjective refraction examination discussed above. For example, during the calibration operation, the lens power required at the first meridian is computed if the measurement provided from the user's eye to the display 140 where the target was displayed was measured in millimeters. The computation involves the following formula:

Meridian1Power=(1/(1stMeridianMMDistance/1000)))*−1 Equation 4.

During examination, the lens power required at the second meridian is computed if the measurement provided from the user's eye to the display 140 where the target was displayed was measured in millimeters. The computation involves the following formula:

Meridian2Power=(1/(2ndMeridianMM1Distance/1000)))*−1 Equation 5.

Additionally the sphere, cylinder, and axis of the final distance prescription (Rx) can be computed using the following formulas and conditions:

Rx Sphere(Use the least minus(most plus) of the 2 meridian powers) Equation 6.
If Meridian1Power=Meridian2Power, then SpherePower=Meridian1Power
If Meridian1Power<Meridian2Power, then SpherePower=Meridian2Power
If Meridian2Power<Meridian1Power then SpherePower=Meridian1Power

Rx Minus Cylinder(Use the difference between Meridian1Power and Meridian2Power) Equation 7.
If Meridian1Power=Meridian2Power then CylinderPower=0 (No cylinder)
If Meridian1Power<Meridian2Power then CylinderPower=Meridian1Power−Meridian2Power
If Meridian2Power<Meridian1Power then CylinderPower=Meridian2Power−Meridian1Power

Rx Minus Cylinder Axis Equation 8.
If Meridian1Power=Meridian2Power then Axis=0 (No axis because Rx has no cylinder)
If Meridian1Power<Meridian2Power then Axis=Meridian1Axis
If Meridian2Power<Meridian1Power then Axis=Meridian2Axis

In some implementations, additional calculations can be performed depending on the age of the user. For example, if the user 102A is a threshold age, e.g., over forty years old, a value can be added to the sphere of the final Rx. In one example, the value is less than 1.0, such as 0.125.

FIGS. 4A-4H illustrate a series of computer-generated graphics 400A-400E shown on the display 140 to be viewed by the user during a second exemplary remote subjective refraction. In the example shown in FIGS. 4A-4H, the examination technique can be used for nearsighted and farsighted patents. Display 140 initially shows interface 400A depicted in FIG. 4A. When the validation object is utilized to calibrate the system, the user 102A receives instructions to hold the validation object (e.g., driver's license, credit card, or some other card-sized document) at a first distance (e.g., one foot) from the center of display 140. The image of the validation object is captured and measured, and measurement in pixels is stored for the user 102A and related to the first distance. Interface 400A displays a calibration box 302A and the user receives instructions to change the vertical and horizontal dimensions of the calibration box 302A until it is the same size as the validation object being to record the vertical and horizontal calibration values.

The user 102A receives instructions to place a chair so that the center of the back of the chair is ten feet and six inches from the center of the display 140. The user's visual acuity is then checked with their current glasses at ten feet. The user 102A receives instructions to sit in the chair while keeping their back against the back of the chair throughout the visual acuity tests.

Display 140 then displays a visual acuity chart. The chart letter sizes are adjusted to the proper sizes for a ten-foot lane using the screen calibration values. The smallest line the user 102A can see while using their current glasses is then determined with their left eye covered. Data indicating the right eye visual acuity is stored locally on the user device 130 or remotely on the server 110, with their current glasses for a ten-foot lane. The smallest line the user 102A can see while using their current glasses with their right eye covered is determined. The smallest line the user 102A can see while using their current glasses with both eyes open is then determined. Data indicating the binocular visual acuity with their current glasses for a 10-foot lane is then stored locally on the user device 130 or remotely on the server 110.

The user's visual acuity without glasses is checked at ten feet. The user 102A receives instructions at the user device 130 to remove their glasses and sit in the chair keeping their back against the back of the chair throughout the visual acuity tests. The visual acuity chart is then displayed. The chart letter sizes are adjusted to the proper sizes for a ten-foot lane using the screen calibration values. The smallest line the user 102A can see without their glasses with their left eye covered. Data indicative of the right eye unaided visual acuity for a 10-foot lane is stored locally on the user device 130 or remotely on the server 110. Data indicating of the smallest line the user 102A can see without their glasses with their right eye covered is stored locally on the user device 130 or remotely on the server 110. Data indicating the left eye unaided visual acuity for a ten-foot lane is then stored locally on the user device 130 or remotely on the server 110. Data indicating the smallest line the user 102A can see while using their current glasses with both eyes open is determined and the binocular unaided visual acuity for a ten-foot lane is stored locally on the user device 130 or remotely on the server 110.

The user's visual acuity with current glasses at four feet is then checked. The user 102A receives instructions to move the chair so the back of the chair is exactly four feet, six inches from the center of the computer monitor. The user 102A receives instructions to sit in the chair keeping their back against the back of the chair throughout the visual acuity tests. The visual acuity chart is then displayed on the display 140 with chart letter sizes that are adjusted to the proper sizes for a four-foot lane using the screen calibration values. The smallest line that the user 102A can see while using their current glasses with their left eye covered is determined. Data indicating the right eye visual acuity is stored locally on the user device 130 or remotely on the server 110, with the user's current glasses for a four-foot lane. The smallest line the user 102A can see while using their current glasses with their right eye covered is then determined. Data indicating the left eye visual acuity is stored locally on the user device 130 or remotely on the server 110, with their current glasses for a four-foot lane. The smallest line the user 102A can see while using their current glasses with both eyes open is determined. The binocular visual acuity with the user's current glasses for a four-foot lane is then determined.

The user's visual acuity without glasses at four feet is also determined by the system 100. User 102A receives instructions to move the chair so the center of the back of the chair is four feet, six inches from the center of the computer monitor. The user 102A receives instructions to remove their glasses and sit in the chair keeping their back against the back of the chair throughout the visual acuity tests. The visual acuity chart is displayed and the letter indicator sizes are adjusted to the proper sizes for a four-foot lane using the screen calibration values. The smallest line the user 102A can see without their glasses with their left eye covered is determined. Data indicating the right eye unaided visual acuity at four feet is stored locally on the user device 130 or remotely on the server 110. The smallest line the user 102A can see without their glasses with their right eye covered is determined. Data indicating the left eye unaided visual acuity at four feet is stored locally on the user device 130 or remotely on the server 110. The smallest line the user 102A can see while using their current glasses with both eyes open is determined. The binocular unaided visual acuity at four feet is determined.

Analyzing examination data enables server 110 to determine whether a prescription is needed. For example, the difference in value of the visual acuity at ten feet compared to the visual acuity at four feet will indicates if the user 102A needs more plus power or more minus power in their current prescription in each eye. The visual acuity at ten feet compared to a normal 20/20 visual acuity will indicates the spherical equivalent of the extra power the user 102A needs in each eye compared to their current prescription. The user 102A needs about 0.25 power change for each line they can read above the 20/20 line. For example, if the best the user 102A can see is only 3 lines above the 20/20 line on the visual acuity chart, the user 102A will need a spherical equivalent change of approximately 0.75 diopter.

The axis for each eye is checked at four feet while the user 102A is still at the four-foot distance with their current glasses on display one of the many axis determination charts. The axis test can be repeated multiple times using different axis charts to help confirm the axis, or in case the user 102A is not responsive to one or more of the axis charts. For example, the user 102A can be instructed to cover their left eye. As the axis chart is rotated, the user 102A can receive a prompt, at the user device 130, instructing the user 102A to find the point in the rotation where the axis indicator is the clearest using their right eye. If the server 110 receives data indicating that the user 102A determined that the axis indicator is the same no matter where it is rotated using each axis determination chart, the server 110 determines that the user 102A does not need their axis or cylinder changed in their current glasses for the right eye. If an axis is located, data indicating the axis is stored locally on the user device 130 or remotely on the server 110, for the right eye at the four-foot distance. The user 102A is then instructed to cover their right eye. As the axis chart is rotated, the user 102A will receive a prompt, at the user device 130, to find the point in the rotation where the axis indicator is the clearest using their left eye.

If the server 110 receives data indicating that the user 102A determined that the axis indicator is the same no matter where it is rotated using each axis determination chart, the server 110 determines that the user 102A does not need their axis or cylinder changed in their current glasses for the left eye. If an axis is located, data indicating the axis for the left eye is stored locally on the user device 130 or remotely on the server 110, at the four-foot distance. The axis is checked for each eye at ten feet. The user 102A receives instructions to move the chair back to the ten-foot, six-inch distance and one of several axis determination charts is displayed with the user's current glasses on. The axis test can be repeated multiple times using different axis charts to help confirm the axis, or in case the user 102A is not responsive to one or more of the axis charts. For example, the user 102A receives a prompt at the user device 130 to cover their left eye. As the axis chart is rotated, the user 102A receives instructions to find the point in the rotation where the axis indicator is the clearest using their right eye.

If the server 110 receives data indicating that the user 102A determined that the axis indicator is the same no matter where it is rotated using each axis determination chart, the server 110 determines that the user 102A does not need their axis or cylinder changed in their current glasses for the right eye. If an axis is located, data indicating the axis for the right eye at the ten-foot distance is stored locally on the user device 130 or remotely on the server 110. The user 102A receives instructions to cover their right eye. As the axis chart is rotated, the user 102A receives instructions to find the point in the rotation where the axis indicator is the clearest using their left eye. If the server 110 receives data indicating that the user 102A determined that the axis indicator is the same no matter where it is rotated using each axis determination chart, the server 110 determines that the user 102A does not need their axis or cylinder changed in their current glasses for the left eye.

If an axis is located, data indicating the axis for the left eye is stored locally on the user device 130 or remotely on the server 110, at the ten-foot distance. Using the information gathered so far, the power of the sphere, cylinder, and axis that is determined may be needed over their current prescription. If only sphere power is needed at this point (no axis was found), then only the spherical equivalent is needed over their current prescription. If an axis is found, then additional testing may be required to determine how much of the power needed over their current prescription is sphere and how much of it is cylinder. Once the additional power is determined, it can be mathematically added as an over-refraction to their current prescription to determine the new prescription. In some instances, such as where an accurate power cannot be determined or that the resulting axis or cylinder power is too great, a remote comprehensive exam may be recommend to the user.

The described systems, methods, and techniques can be implemented in digital electronic circuitry, computer hardware, firmware, software, or in combinations of these elements. Apparatus implementing these techniques can include appropriate input and output devices, a computer processor, and a computer program product tangibly embodied in a machine-readable storage device for execution by a programmable processor. A process implementing these techniques can be performed by a programmable processor executing a program of instructions to perform desired functions by operating on input data and generating appropriate output. The techniques can be implemented in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Suitable processors include, by way of example, both general and special purpose microprocessors. Generally, a processor will receive instructions and data from a read-only memory and/or a random-access memory. Storage devices suitable for tangibly embodying computer program instructions and data include all forms of non-volatile memory, including by way of example semiconductor memory devices, such as Erasable Programmable Read-Only Memory (EPROM), Electrically Erasable Programmable Read-Only Memory (EEPROM), and flash memory devices; magnetic disks such as internal hard disks and removable disks; magneto-optical disks; and Compact Disc Read-Only Memory (CD-ROM). Any of the foregoing can be supplemented by, or incorporated in, specially designed application-specific integrated circuits (ASICs).

It will be understood that various modifications can be made. For example, other useful implementations could be achieved if operations of the disclosed techniques were performed in a different order and/or if components in the disclosed systems were combined in a different manner and/or replaced or supplemented by other components. Accordingly, other implementations are within the scope of the disclosure.

1. A method of enabling a remote vision examination for a user over a wide area network, the method comprising:
obtaining, from one or more user devices, calibration data relating to a user positioning for the remote vision examination;
determining an egocentric distance between the user and the one or more user devices based on the calibration data;
determining that a set of criteria associated with the remote vision examination have been satisfied at least on based on the egocentric distance and the calibration data;
in response to determining that the set of criteria have been satisfied, transmitting, to a provider device, an instruction that permits a vision service provider to administer the remote vision examination;
establishing, over the wide area network, a communication session between the one or more user devices and at the provider device;
collecting examination data generated during the remote vision examination; and
providing, to the provider device, the examination data in a manner that permits the vision service provider to at least evaluate the examination data.

2. The method of claim 1, wherein:
the one or more user devices comprises a computing device that displays information relating to remote vision examination and a camera for collecting video data of the user during the remote vision examination; and
the calibration data comprises:
a focal length of a camera; and
one or more distortion coefficients associated with radial distortion of a lens of the camera.

3. The method of claim 1, wherein the calibration data comprises a size associated with a reference object.

4. The method of claim 1, wherein the examination data comprises information associated with a subjective refraction test.

5. The method of claim 1, wherein:
the examination data comprises information related to a subjective refraction; and
transmitting the examination data to the provider device permits the vision service provider to determine a best-corrected visual acuity for the user based on the examination data.

6. The method of claim 1, wherein the remote vision examination comprises a visual acuity test.

7. The method of claim 1, wherein:
the one or more user devices comprises a computing device that displays information relating to remote vision examination and a camera for collecting video data of the user during the remote vision examination; and
the set of criteria comprises:
a physical coupling the computing device and the camera;
a user device configuration in which a sensor of the computing device is on a same plane as a display of the computing device;
a network connection speed having an upload or download speed of at least about 50 Mbps; and
a minimum ambient light level of about 500 lux.

8. The method of claim 1, wherein the set of criteria comprises:
a trained reference object detector capable of facial landmark recognition is installed on a server coupled to the wide area network.

9. The method of claim 8, further comprising:
determining that information specified by the calibration data does not satisfy one or more criteria included in the set of criteria;
in response to determining that the information does not satisfy the one or more criteria, determining a set of actions to be performed by the user;
obtaining, from the one or more user devices, second calibration data, wherein the second calibration data indicates that the user has performed the set of actions; and
determining that information specified by the second calibration data satisfies the set of criteria.

10. The method of claim 1, comprising:
in response to determining the egocentric distance, providing, to the one or more user devices, an instruction to display one or more optotypes having a width and height that corresponds to the egocentric distance.

11. The method of claim 10, further comprising:
determining a pixel density of the a display included in the one or more user devices;
determining a minim angle of resolution for the one or more optotypes for display on the display; and
transmitting, to the provider device, information related to a calibration of the display.

12. A system comprising:
one or more computing devices; and
one or more storage devices that store executable instructions that, when executed by the one or more computing devices, cause the one or more computing devices to perform operations comprising:
obtaining, from one or more user devices, calibration data relating to a user positioning for the remote vision examination;
determining an egocentric distance between the user and the one or more user devices based on the calibration data;
determining that a set of criteria associated with the remote vision examination have been satisfied at least on based on the egocentric distance and the calibration data;
in response to determining that the set of criteria have been satisfied, transmitting, to a provider device, an instruction that permits a vision service provider to administer the remote vision examination;
establishing, over the wide area network, a communication session between the one or more user devices and at the provider device;
collecting examination data generated during the remote vision examination; and
providing, to the provider device, the examination data in a manner that permits the vision service provider to at least evaluate the examination data.

13. The system of claim 12, wherein:
the one or more user devices comprises a computing device that displays information relating to remote vision examination and a camera for collecting video data of the user during the remote vision examination; and
the calibration data comprises:
a focal length of a camera; and
one or more distortion coefficients associated with radial distortion of a lens of the camera.

14. The system of claim 12, wherein the calibration data comprises a size associated with a reference object.

15. The system of claim 12, wherein the examination data comprises information associated with a subjective refraction test.

16. The system of claim 12, wherein:
the examination data comprises information related to a subjective refraction; and
transmitting the examination data to the provider device permits the vision service provider to determine a best-corrected visual acuity for the user based on the examination data.

17. At least one non-transitory computer-readable storage device storing instructions that, when executable by one or more processors, cause the one or more processors to perform operations comprising:
obtaining, from one or more user devices, calibration data relating to a user positioning for the remote vision examination;
determining an egocentric distance between the user and the one or more user devices based on the calibration data;
determining that a set of criteria associated with the remote vision examination have been satisfied at least on based on the egocentric distance and the calibration data;
in response to determining that the set of criteria have been satisfied, transmitting, to a provider device, an instruction that permits a vision service provider to administer the remote vision examination;
establishing, over the wide area network, a communication session between the one or more user devices and at the provider device;
collecting examination data generated during the remote vision examination; and
providing, to the provider device, the examination data in a manner that permits the vision service provider to at least evaluate the examination data.

18. The storage device of claim 17, wherein:
the one or more user devices comprises a computing device that displays information relating to remote vision examination and a camera for collecting video data of the user during the remote vision examination; and
the calibration data comprises:
a focal length of a camera; and
one or more distortion coefficients associated with radial distortion of a lens of the camera.

19. The storage device of claim 17, wherein the calibration data comprises a size associated with a reference object.

20. The storage device of claim 17, wherein the examination data comprises information associated with a subjective refraction test.
